(19) United States
(12) Patent Application Publication
(10) Pub. No.: US 2025/0051358 A1
(43) Pub. Date: Feb. 13, 2025

(54) PROCESSES FOR THE PREPARATION AND PURIFICATION OF GPR119 AGONIST COMPOUNDS AND INTERMEDIATES THEREOF

(71) Applicant: MANKIND PHARMA LTD., New Delhi (IN)

(72) Inventors: Sazid ALI, Gurugram (IN); Rakesh Ishwar PATIL, Gurugram (IN); Santosh Kumar RAI, Gurugram (IN); Anil KUMAR, Gurugram (IN)

(21) Appl. No.: 18/799,812

(22) Filed: Aug. 9, 2024

Related U.S. Application Data
(63) Continuation of application No. PCT/IB2024/057711, filed on Aug. 8, 2024.

(30) Foreign Application Priority Data
Aug. 11, 2023 (IN) 202311054017

(51) Int. Cl. C07D 513/04

(52) U.S. Cl. CPC C07D 513/04

(57) ABSTRACT

The present disclosure relates to improved processes for the preparation and purification of a compound of Formula (I) and pharmaceutically acceptable salts thereof, and its intermediates.

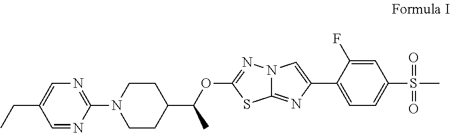 [SMILES: CCc1cnc(N2CCC([C@H](C)Oc3nn4cc(-c5ccc(S(C)(=O)=O)cc5F)nc4s3)CC2)nc1]

The present application is a continuation of International Patent Application No. PCT/IB2024/057711, filed Aug. 8, 2024, which claims the benefit of Indian Patent Application No. IN 202311054017, filed on Aug. 11, 2023, each of which is hereby incorporated by reference.

### FIELD OF THE INVENTION

The present invention relates to improved processes for the preparation and purification of GPR119 agonist compounds, such as a compound of Formula (I) and pharmaceutically acceptable salts thereof, and its intermediates. The present invention also relates to substantially pure GPR119 agonist compounds, such as a substantially pure compound of Formula I or a pharmaceutically acceptable salt thereof, and to uses thereof

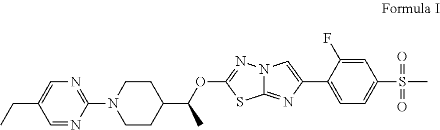 [SMILES: CCc1cnc(N2CCC([C@H](C)Oc3nn4cc(-c5ccc(S(C)(=O)=O)cc5F)nc4s3)CC2)nc1]

### BACKGROUND OF THE INVENTION

There are many drugs useful for the treatment of conditions such as diabetes mellitus, including GPR119 agonists. GPR119 agonists mediate a unique nutrient-dependent dual elevation of both insulin and glucagon like peptide glucose-dependent insulinotropic peptide levels in vivo. As a stand-alone therapy or in tandem with approved DPP-IV inhibitors, they could herald a new treatment paradigm for type 2 diabetes mellitus. Activation of GPR119 has been demonstrated to stimulate intracellular cAMP and lead to glucose dependent GLP-1 and insulin secretion.

U.S. Pat. No. 10,208,030 describes GPR119 agonists, including 2-((S)-1-(1-(5-ethylpyrimidin-2-yl) piperidin-4-yl) ethoxy)-6-(2-fluoro-4-(methylsulfonyl)phenyl) imidazo [2,1-b][1,3,4]thiadiazole, and processes for their preparation.

There is, however, a need for improved processes for the preparation of 2-((S)-1-(1-(5-ethylpyrimidin-2-yl.) piperidin-4-yl) ethoxy)-6-(2-fluoro-4-(methylsulfonyl)phenyl) imidazo [2,1-b][1,3,4]thiadiazole and pharmaceutically acceptable salts thereof (and its intermediates).

### SUMMARY OF THE INVENTION

In one aspect, the present invention relates to a process for the preparation of a compound of Formula I or pharmaceutically acceptable salt thereof

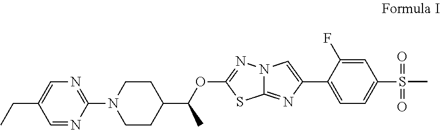 [SMILES: CCc1cnc(N2CCC([C@H](C)Oc3nn4cc(-c5ccc(S(C)(=O)=O)cc5F)nc4s3)CC2)nc1]

the process comprising:
(a) reacting a compound of Formula III-a or a salt thereof with a compound of Formula IV-a or a salt thereof to obtain a compound of Formula II-a or a salt thereof (e.g., a pharmaceutically acceptable salt thereof)

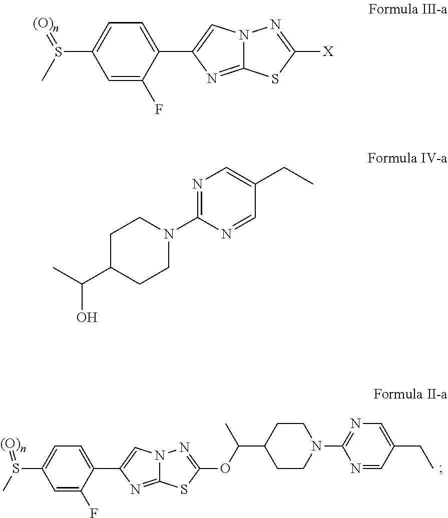

wherein X is a leaving group and n is 0, 1 or 2;
(b) when n is 0 or 1, converting the compound of Formula II-a to compound of Formula I-a or a pharmaceutically acceptable salt thereof

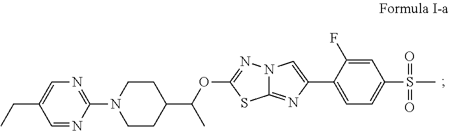 [SMILES: CCc1cnc(N2CCC(C(C)Oc3nn4cc(-c5ccc(S(C)(=O)=O)cc5F)nc4s3)CC2)nc1]

(c) separating the desired isomer of the compound of Formula I or a pharmaceutically acceptable salt thereof; and
(d) optionally purifying the desired isomer of the compound of Formula I or a pharmaceutically acceptable salt thereof. The compounds of Formula II-a and IV-a may be racemic. The compound of formula I-a may be racemic, such as when the compound of formula II-a is racemic. In one embodiment, steps (a)-(d) are performed sequentially.

In another aspect, the present invention relates to a process for the preparation of a compound of Formula I or a pharmaceutically acceptable salt thereof

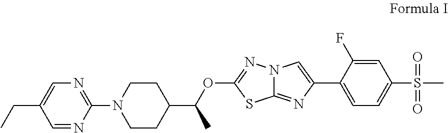 [SMILES: CCc1cnc(N2CCC([C@H](C)Oc3nn4cc(-c5ccc(S(C)(=O)=O)cc5F)nc4s3)CC2)nc1]

the process comprising:
(a) reacting a compound of Formula III-1 or a salt thereof with a compound of Formula IV or a salt thereof to obtain a compound of Formula II or a salt thereof

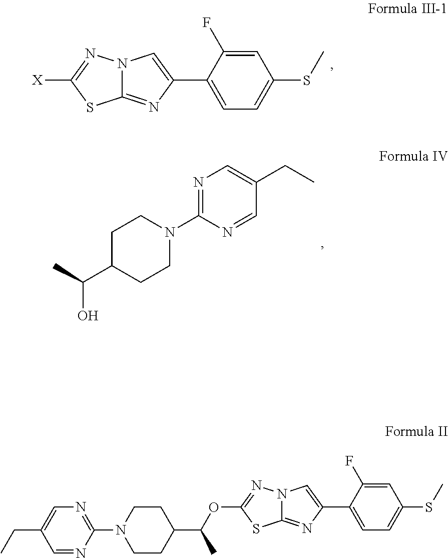

wherein X is a leaving group;
(b) converting the compound of Formula II or a salt thereof to a compound of Formula I or pharmaceutically acceptable salt thereof; and
(c) optionally purifying the compound of Formula I or a pharmaceutically acceptable salt thereof.

In one embodiment, the compound of Formula III-1 or a salt thereof is prepared by a process comprising:
(a) reacting a compound of Formula VIII with an acetyl halide to form a compound of Formula VII

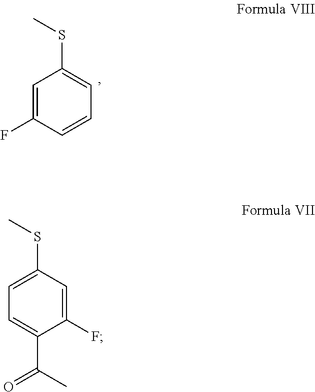

(b) converting the compound of Formula VII to a compound of Formula V-1 in the presence of solvent and a halogenating agent

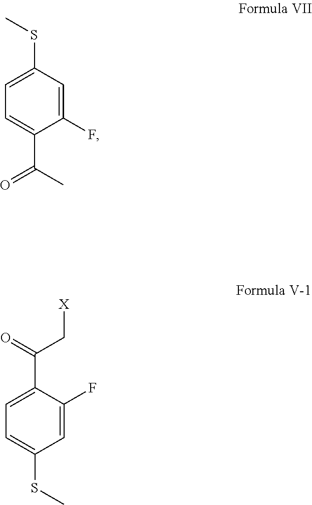

wherein X is a leaving group (such as a halogen containing leaving group, e.g., Cl, Br, and I);
(c) reacting the compound of Formula V-1 with a compound of Formula VI-1 or a salt thereof to give a compound of Formula III-1 or a salt thereof

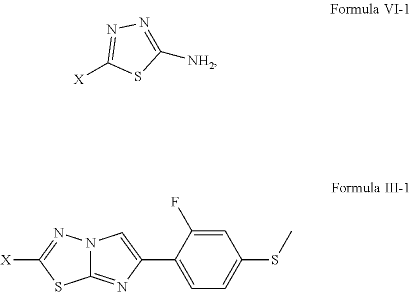

wherein X is as defined above; and
(d) optionally, purifying the compound of Formula III-1 or a salt thereof using one or more suitable solvents.

In one embodiment, the acetyl halide is selected from acetyl fluoride, acetyl chloride, acetyl bromide, acetyl iodide, and any combination of any of the foregoing, In one embodiment, the acetyl halide is acetyl chloride.

In one embodiment, the halogenating agent is selected from fluorine, chlorine, bromine, iodine, N-chloroosuccinimide, N-bromosuccinimide, N-iodosuccinimide, hydrogen fluoride, hydrogen chloride, hydrogen bromide, hydrogen iodide, thionyl chloride, thionyl bromide, oxalyl chloride, oxalyl bromide, and any combination thereof.

In one embodiment, the compound of Formula IV or a salt thereof is prepared by a process comprising:
(a) reacting a compound of Formula XI with a reducing agent to give a compound of formula X

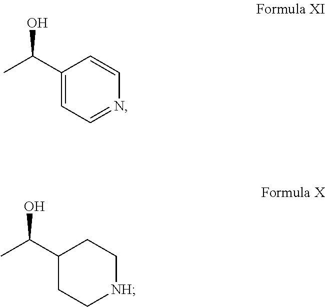

(b) reacting the compound of Formula X with a compound of Formula IX-a or a salt thereof, where X is a leaving group (such as a halogen, e.g., Cl) in the presence of solvent to obtain a compound of Formula IV

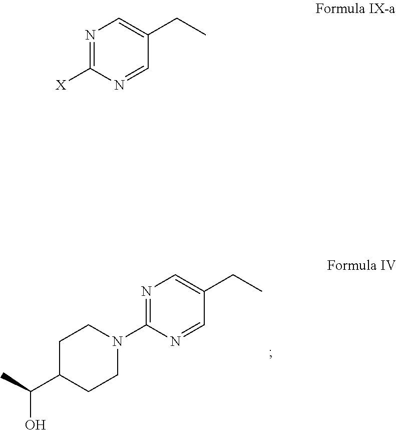

and
(c) optionally purifying the compound of Formula IV.

In one embodiment, the reducing agent is selected from Ni, Raney Ni, Pd/C, Pd(OH)₂, Na metal, Pt, PtO₂ and any combination thereof.

In one embodiment, the process for the preparation of a compound of Formula I or a pharmaceutically acceptable salt thereof comprises:
(a) reacting a hydrobromide salt of a compound of Formula III with a compound of Formula IV or a salt thereof to give a compound of Formula II-b or a salt thereof in racemic form

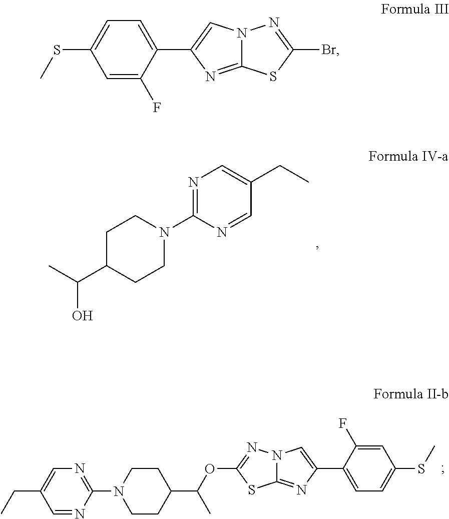

(b) converting the compound of Formula II-b or a salt thereof to a compound of Formula I-a or a pharmaceutically acceptable salt thereof in racemic form

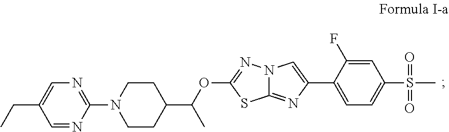 [SMILES: CCc1cnc(N2CCC(C(C)Oc3nn4cc(-c5ccc(S(C)(=O)=O)cc5F)nc4s3)CC2)nc1]

(c) separating the desired isomer of the compound of Formula I or pharmaceutically acceptable salt thereof; and
(d) optionally purifying the desired isomer of the compound of Formula I or pharmaceutically acceptable salt thereof. In one embodiment, the compounds of formulas IV-a, II-b, and I-a are in racemic form.

In one embodiment, step (c) comprises
(i) adding a chiral acid to the compound of Formula I-a or a pharmaceutically acceptable salt thereof (e.g., where the compound of Formula I-a or its salt is in racemic form) in one or more solvents;
(ii) optionally isolating the chiral acid salt of the compound of Formula I;
(iii) adding a base;
(iv) separating the desired isomer of the compound of Formula I or a pharmaceutically acceptable salt thereof; and
(v) optionally purifying the desired isomer of the compound of Formula I or a pharmaceutically acceptable salt thereof.

In one embodiment, step (c) is performed by chiral chromatography using a solvent or solvent mixture selected from dichloromethane, methanol, n-hexane, n-heptane, ethanol (EtOH), isopropanol (IPA), tetrahydrofuran (THF), acetonitrile (ACN), ethyl acetate (EtOAc), MTBE, n-butanol, and any combination of any of the foregoing as an eluent.

In another aspect, the present invention relates to a process for the purification of a compound of Formula I or a pharmaceutically acceptable salt thereof

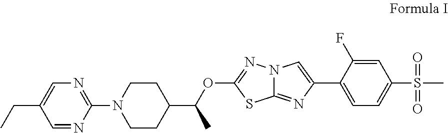 [SMILES: CCc1cnc(N2CCC([C@H](C)Oc3nn4cc(-c5ccc(S(C)(=O)=O)cc5F)nc4s3)CC2)nc1]

the process comprising:
(a) providing a solution, a dispersion, or a slurry of the compound of Formula I or a pharmaceutically acceptable salt thereof in one or more solvents;
(b) heating the reaction mass of step (a);
(c) cooling the reaction mass;
(d) optionally adding one or more solvents and/or one or more antisolvents; and
(e) isolating the purified compound of Formula I or a pharmaceutically acceptable salt thereof.

In one embodiment, the one or more solvents are selected from methanol, ethanol, acetonitrile, dimethyl sulfoxide, cyclohexane, dichloromethane, and any combination of any of the foregoing.

In another aspect, the present invention relates to a compound of Formula I or a pharmaceutically acceptable salt thereof

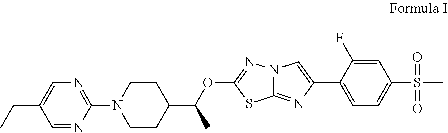 [SMILES: CCc1cnc(N2CCC([C@H](C)Oc3nn4cc(-c5ccc(S(C)(=O)=O)cc5F)nc4s3)CC2)nc1]

where the compound of Formula I or a pharmaceutically acceptable salt thereof has
(i) a chemical purity of more than about 99%,
(ii) an enantiomeric excess of more than about 99%, or
(iii) both (i) and (ii).

In another aspect, the present invention relates to a compound of Formula I or a pharmaceutically acceptable salt thereof

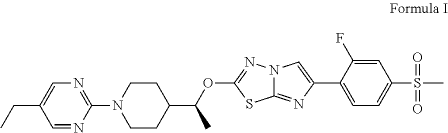 [SMILES: CCc1cnc(N2CCC([C@H](C)Oc3nn4cc(-c5ccc(S(C)(=O)=O)cc5F)nc4s3)CC2)nc1]

wherein the compound of Formula I or a pharmaceutically acceptable salt thereof is substantially free (e.g., contains less than about 0.2%, 0.1%, 0.05%, 0.02%, or 0.01% w/w) of one or more compounds of Formula A, B, C, D, and E (e.g., substantially free of each compound of Formula A, B, C, D, and E).

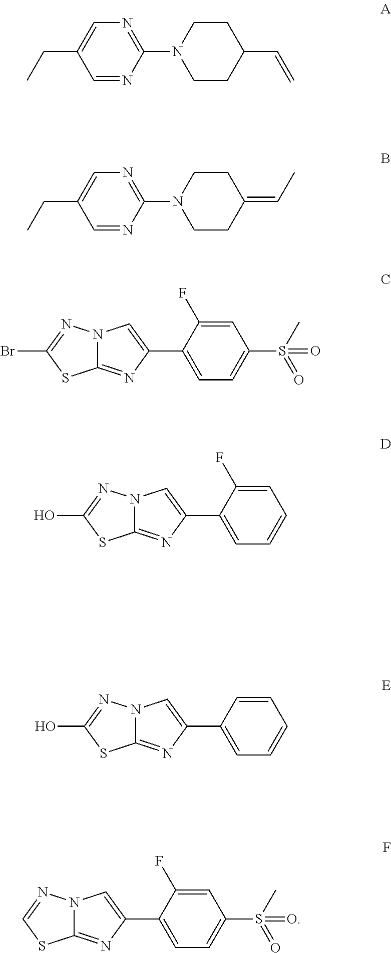

In one embodiment, the compound of Formula I or salt thereof have (i) a d₉₀ of less than about 60 μm, (ii) a d₅₀ of less than about 20 μm, (iii) a d₁₀ of less than about 10 μm, or (iv) any combination of any of (i), (ii) and (iii).

In another aspect, the present invention relates to a compound of Formula I or a pharmaceutically acceptable salt thereof

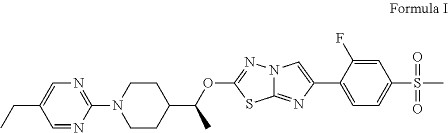 [SMILES: CCc1cnc(N2CCC([C@H](C)Oc3nn4cc(-c5ccc(S(C)(=O)=O)cc5F)nc4s3)CC2)nc1]

wherein the compound of Formula I or a pharmaceutically acceptable salt thereof is characterized by a particle size distribution having
(i) a d₉₀ of less than about 60 μm,
(ii) a d₅₀ of less than about 20 μm,
(iii) a d₁₀ of less than about 10 μm, or
(iv) any combination of (i), (ii) and (iii).

### DETAILED DESCRIPTION OF THE INVENTION

As used herein, the term “substantially free” means a compound of formula I having one or more compounds of Formulas A, B, C, D, and E present in less than about 0.3% by area percentage of HPLC, such as less than about 0.2% by area percentage of HPLC, or less than about 0.15%, 0.1%, 0.05%, 0.02%, or 0.01% by area percentage of HPLC. In one embodiment, the compound of formula I is free of one or more compounds of Formulas A, B, C, D, and E, i.e., one or more compounds of Formulas A, B, C, D, and E are not present in a detectable amount by area percentage of HPLC.

As used herein, the term “substantially pure” refers to the chemical purity of a compound which is at least about 85%, at least about 90%, at least about 95.0%, at least about 98%, at least about 99%, at least about 99.5% or at least about 99.9%, as measured by a HPLC.

The compound of Formula I is also known as 2-((S)-1-(1-(5-ethyl-pyrimidin-2-yl) piperidin-4-yl) ethoxy)-6-(2-fluoro-4-(methylsulfonyl)phenyl)-imidazo [2,1-b][1,3,4]thiadiazole.

Any of the salts of any of the compounds herein (such as the intermediate compounds) can be pharmaceutically acceptable salts.

Suitable pharmaceutically acceptable salts (or salts) for use in the present invention may be, but are not limited to, salts of inorganic acids, such as, e.g., hydrochloric acid, hydrobromic acid, sulphuric acid, phosphoric acid salt; salts of organic acids such as, e.g., succinic acid, formic acids, acetic acid, diphenyl acetic acid, triphenylacetic acid, caprylic acid, dichloroacetic acid, trifluoro acetic acid, propionic acid, butyric acid, lactic acid, citric acid, gluconic acid, mandelic acid, tartaric acid, malic acid, adipic acid, aspartic acid, fumaric acid, glutamic acid, maleic acid, malonic acid, benzoic acid, p-chlorobenzoic acid, nicotinic acid, o-hydroxybenzoic acid, p-hydroxybenzoic acid, 1-hydroxy-naphthalene-2-carboxylic acid, hydroxynaphthalene-2-carboxylic acid, ethanesulfonic acid, ethane-1,2-disulfonic acid, 2-hydroxyethane sulfonic acid, methanesulfonic acid, (+)-camphor-10-sulfonic acid, benzenesulfonic acid, naphthalene-2-sulfonic acid, p-toluenesulfonic acid; salts of pharmaceutically acceptable bases such as, e.g., metal salts including alkali metal or alkaline earth metal salts such as sodium, potassium, magnesium, calcium and zinc salts, ammonium salts; and any combination of any of the foregoing.

As used herein, the term “suitable solvent” or “solvent” includes solvents that may be used for preparing a compound of Formula I or a pharmaceutically acceptable salt thereof (and its intermediates) and may be selected from, but not limited to, C₁-C₆ alcohols, C₁-C₈ hydrocarbons, halogenated hydrocarbons, ethers, C₃-C₈ ketones, esters, nitriles, sulphonamides, acetamides, pyrrolidines, formamides, water and mixture of any of the foregoing. Examples include, but are not limited to methanol, ethanol, butanol, t-butanol, isopropyl alcohol, n-propyl alcohol, iso-butanol, pentanol, glycols, toluene, chlorobenzene, acetonitrile, dimethyl acetamide (DMA), dimethylformamide (DMF), N-methyl pyrrolidine (NMP), dimethyl sulfoxide (DMSO), hexamethyl phosphoramide (HMPA), tetrahydrofuran (THF), methyl tetrahydrofuran, dioxane, acetone, methyl ethyl ketone (MEK), methyl isobutyl ketone (MIBK), methyl t-butyl ketone, dichloromethane, dichloroethane, chloroform, tetrachloromethane, chlorobenzene, ethyl acetate, propyl acetate, propenyl acetate, t-butyl acetate, hexane, n-heptane, cyclohexane, petroleum benzine, water and any combination of any of the foregoing.

As used herein, the term “suitable base” or “base” includes bases that may be used for preparing a compound of Formula I or a pharmaceutically acceptable salt thereof (and its intermediates), for purification, and for crystallization and may be selected from, but not limited to, alkali metal or alkaline earth metal hydrides, hydroxides, bicarbonates, carbonates, and any combination of the foregoing. Examples include, but are not limited to, sodium hydride, sodium hydroxide, sodium bicarbonate, sodium carbonate, lithium hydroxide, potassium hydroxide, potassium bicarbonate, potassium carbonate, caesium hydroxide, caesium carbonate, magnesium carbonate, magnesium hydroxide, ammonia, ammonium hydroxide, alkyl amines such as methylamine, ethylamine, dimethylamine, diethylamine, diisopropylamine, triethylamine, trimethylamine; and any combination of any of the foregoing.

As used herein, the term “reaction mixture” or “reaction mass” includes, but is not limited to, a clear solution, a partially dissolved solution, a suspension, a slurry, a turbid solution, a mixture, a biphasic solution or any other phase as known in the literature.

The present invention will now be explained in more detail. While the invention is susceptible to various modifications and alternative forms, specific embodiments thereof are described in detail below. It should be understood, however that it is not intended to limit the invention to the particular forms disclosed, but on the contrary, the invention is to cover all modifications, equivalents, and alternatives falling within the scope of the invention.

The steps of a method may be providing more details that are pertinent to understanding the embodiments of the present invention and so as not to obscure the disclosure with details that will be readily apparent to those of ordinary skill in the art having benefit of the description herein.

The salt of any intermediate (such as the compound of formula II, II-a, II-b, III, III-a, III-1, IV, IV-a, V-1, VI-1, VII, VIII, IX-a, X, and XI) described herein can be a pharmaceutically acceptable salt of the intermediate.

In a first aspect, the present invention relates to a process for the preparation of a compound of Formula I

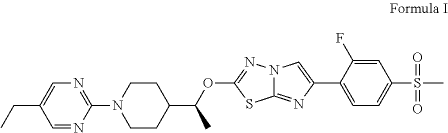 [SMILES: CCc1cnc(N2CCC([C@H](C)Oc3nn4cc(-c5ccc(S(C)(=O)=O)cc5F)nc4s3)CC2)nc1]

or a pharmaceutically acceptable salt thereof.

In a first embodiment, the process of the first aspect comprises:
(a) reacting a compound of Formula III-1 or a salt thereof with a compound of Formula IV or a salt thereof to obtain a compound of Formula II or a salt thereof

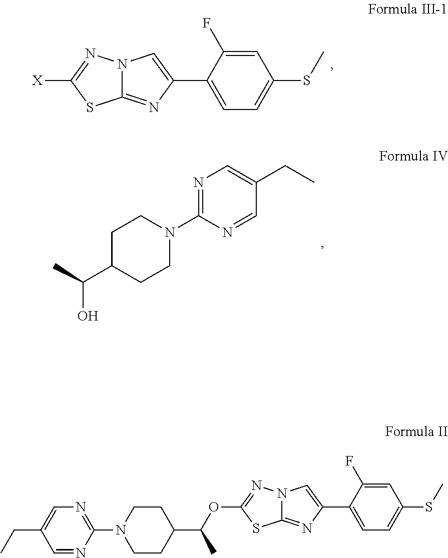

wherein
X is a leaving group, e.g., a leaving group selected from halogen (e.g., fluorine (—F), chlorine (—CI), bromine (—Br), iodine (—I)), mesylate (—OMs), tosylate (—OTs), triflate (—OTf), dinitrogen (—N₂⁺), a dialkylether (—OR₂⁺), a thioether (—SR₂⁺), an amine (—NR₃⁺), ammonia (—NH₃⁺), nitrate (—ONO₂), a phosphate (—OPO(OH)₂), a carboxylate (—OCOR), a phenoxide (—OAr), hydroxide (—OH), an alkoxide (—OR), water (—OH₂⁺), and an alcohol (—OHR⁺), wherein each R group is independently selected from H and C₁₋₃ alkyl and Ar is selected from unsubstituted phenyl or phenyl substituted with one or more substituents selected from halogen, C₁₋₃ alkyl, and any combination of any of the foregoing;
(b) converting the compound of Formula II or a salt thereof to a compound of Formula I or a pharmaceutically acceptable salt thereof (e.g., by oxidizing the compound of Formula II using a suitable oxidizing agent such as oxone); and
(c) optionally purifying the compound of Formula I or a pharmaceutically acceptable salt thereof (e.g., by using one or more suitable solvents as described herein).

In a second embodiment, the process of the first aspect comprises:
(a) reacting a compound of Formula III or a thereof with a compound of Formula IV or a salt thereof to obtain compound of Formula II or a pharmaceutically acceptable salt thereof

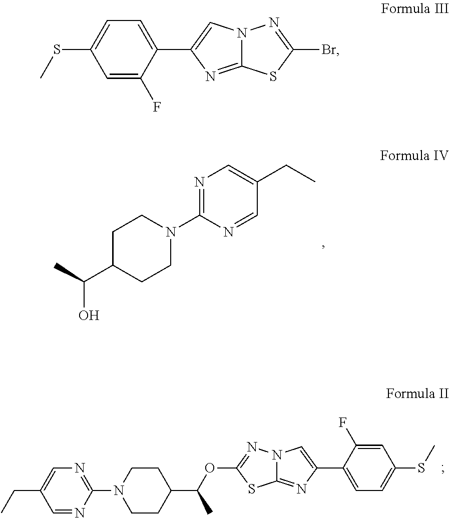

(b) converting the compound of Formula II or a salt thereof to a compound of Formula I or a pharmaceutically acceptable salt thereof (e.g., by oxidizing the compound of Formula II using a suitable oxidizing agent as oxone); and
(c) optionally purifying the compound of Formula I or a pharmaceutically acceptable salt thereof (e.g., by using one or more suitable solvents as described herein).

In a third embodiment, the process of the first aspect comprises:
(a) reacting a compound of Formula VIII with an acetyl halide to obtain compound of formula VII,

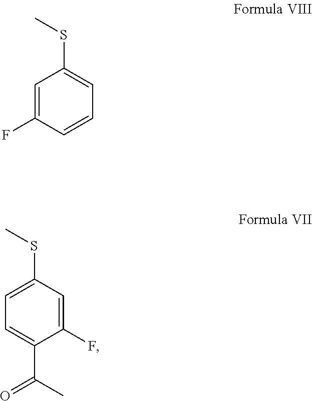

(b) converting the compound of Formula VII to a compound of Formula V-1 or a salt thereof in the presence of a solvent and a halogenating agent

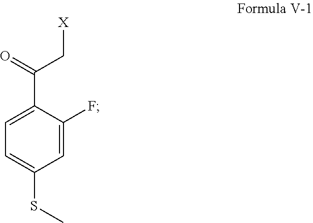

(c) reacting the compound of Formula V-1 or a salt thereof with a compound of formula VI-1 or a salt thereof to obtain a compound of Formula III-1 or a salt thereof

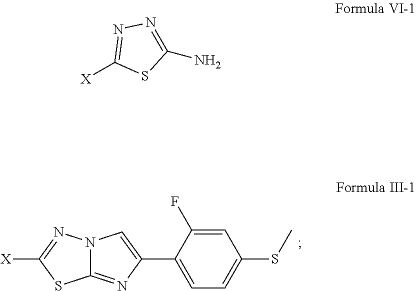

(d) optionally purifying the compound of Formula III-1 or a salt thereof (e.g., by using one or more suitable solvents as described herein); and
(e) converting the compound of Formula III-1 or a salt thereof to a compound of Formula I or a pharmaceutically acceptable salt thereof (e.g., by coupling a compound of Formula III-I (e.g., where X is Br) with a compound of Formula IV),
   wherein
each occurrence of X is independently a leaving group (e.g., a leaving group selected from halogen (e.g., fluorine (—F), chlorine (—CI), bromine (—Br), iodine (—I)), mesylate (—OMs), tosylate (—OTs), triflate (—OTf), dinitrogen (—N₂⁺), a dialkylether (—OR₂⁺), a thioether (—SR₂⁺), an amine (—NR₃⁺), ammonia (—NH₃⁺), nitrate (—ONO₂), a phosphate (—OPO(OH)₂), a carboxylate (—OCOR), a phenoxide (—OAr), hydroxide (—OH), an alkoxide (—OR), water (—OH₂⁺), and an alcohol (—OHR⁺), wherein each R group is independently selected from H and C₁₋₃ alkyl and Ar is selected from unsubstituted phenyl or phenyl substituted with one or more substituents selected from halogen, C₁₋₃ alkyl, and any combination thereof.

In one embodiment, in step (a), the acetyl halide is selected from the group consisting of acetyl fluoride, acetyl chloride, acetyl bromide, acetyl iodide, or any combination thereof. In one embodiment, the acetyl halide is acetyl chloride.

In one embodiment, step (b) is conducted in the presence of a halogenating agent selected from the group consisting of fluorine, chlorine, bromine, iodine, N-chloroosuccinimide, N-bromosuccinimide, N-iodosuccinimide, hydrogen fluoride, hydrogen chloride, hydrogen bromide, hydrogen iodide, thionyl chloride, thionyl bromide, oxalyl chloride, oxalyl bromide, and any combination of any of the foregoing.

In a fourth embodiment, the process of the first aspect comprises:
(a) reacting a compound of Formula VIII with acetyl chloride to obtain a compound of formula VII,

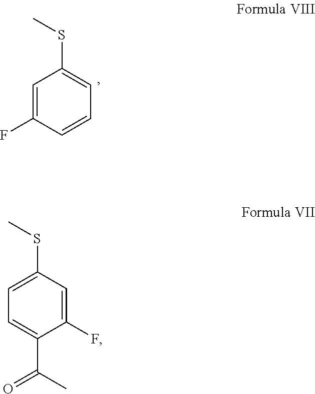

(b) reacting the compound of Formula VII with a brominating agent in the presence of a solvent to obtain a compound of Formula V,

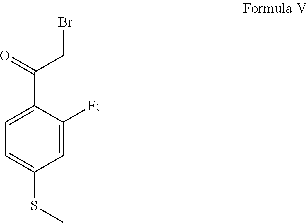

(c) reacting the compound of Formula V with a compound of Formula VI or a salt thereof to obtain a compound of Formula III or a salt thereof

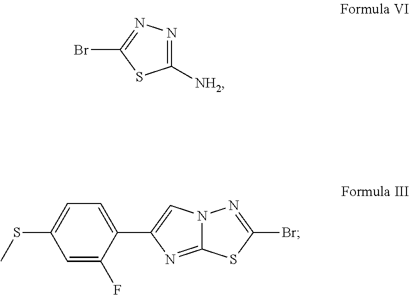 [SMILES: Nc1nnc(Br)s1 ; CSc1ccc(-c2cn3nc(Br)sc3n2)c(F)c1]

(d) optionally purifying the compound of Formula III or a salt thereof (e.g., by using one or more suitable solvents as described herein); and
(e) converting the compound of Formula III or a salt thereof to a compound of Formula I or a pharmaceutically acceptable salt thereof (e.g., by coupling compound of Formula III-I (e.g., where X is Br) with a compound of Formula IV).

In a fifth embodiment, the process of the first aspect comprises:
(a) reacting a compound of Formula XI or a salt thereof with a reducing agent to obtain a compound of formula X or a salt thereof

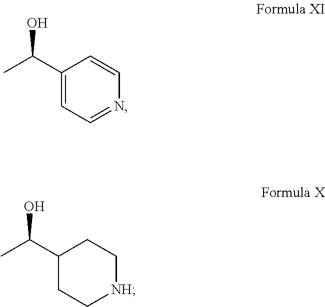

(b) reacting the compound of Formula X or a salt thereof with a compound of Formula IX or a salt thereof in the presence of a solvent to obtain a compound of Formula IV or a salt thereof,

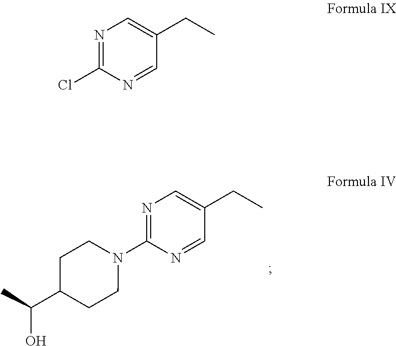

(c) optionally purifying the compound of Formula IV or a salt thereof (e.g., by using one or more suitable solvents as described herein); and
(e) converting the compound of Formula IV or a salt thereof to a compound of Formula I or a pharmaceutically acceptable salt thereof (e.g., by coupling compound of Formula III-I (e.g., where X is Br) with a compound of Formula IV).

In one embodiment, in step (a), the reducing agent is a metallic reducing agent (such as a transition metal catalyst). In one embodiment, the reducing agent is selected from Ni, Raney Ni, Pd/C, Pd(OH)₂, Na metal, Pt, PtO₂, and any combination of any of the foregoing.

In a sixth embodiment, the process of the first aspect comprises:
(a) reacting a compound of Formula III or a salt thereof with a compound of Formula IV-a or a salt thereof (e.g., where the compound of formula IV-a is in racemic form) to obtain a compound of formula II-b or a salt thereof (e.g., where the compound of formula II-b is in racemic form),

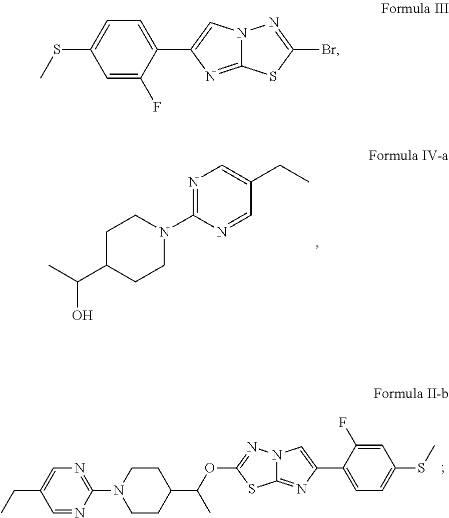

(b) converting the compound of Formula II-b to a compound of Formula I-a (e.g., by oxidizing the compound of Formula II using a suitable oxidizing agent as oxone)

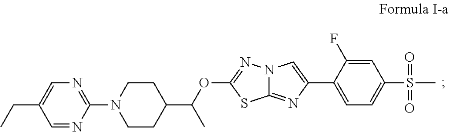 [SMILES: CCc1cnc(N2CCC(C(C)Oc3nn4cc(-c5ccc(S(C)(=O)=O)cc5F)nc4s3)CC2)nc1]

(c) separating the desired isomer of a compound of Formula I or a pharmaceutically acceptable salt thereof (e.g., using chiral chromatography); and
(d) optionally purifying the desired isomer of a compound of Formula I or a pharmaceutically acceptable salt thereof (e.g., by using one or more suitable solvents as described herein).

In one embodiment, the compounds of formulas IV-a, II-b, and I-a are in racemic form.

In a seventh embodiment, the process of the first aspect comprises:
(a) reacting a salt of a compound of Formula III-a with a compound of Formula IV-a or a salt thereof (e.g., where the compound of Formula IV-a or salt thereof is in racemic form) to obtain a compound of Formula II-a or a salt thereof (e.g., where the compound of Formula II-a or salt thereof is in racemic form)

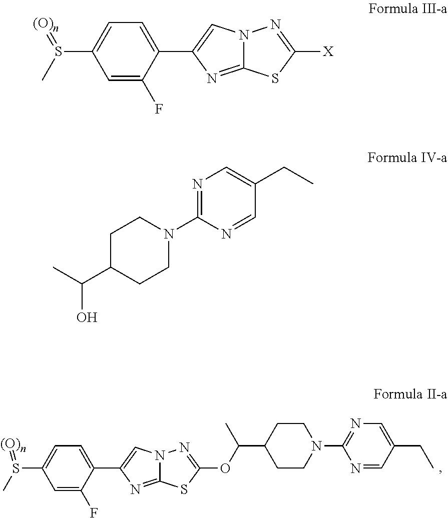

wherein
X is a leaving group (e.g., a leaving group selected from halogen (e.g., fluorine (—F), chlorine (—CI), bromine (—Br), iodine (—I)), mesylate (—OMs), tosylate (—OTs), triflate (—OTf), dinitrogen (—N₂⁺), a dialkylether (—OR₂⁺), a thioether (—SR₂⁺), an amine (—NR₃⁺), ammonia (—NH₃⁺), nitrate (—ONO₂), a phosphate (—OPO(OH)₂), a carboxylate (—OCOR), a phenoxide (—OAr), hydroxide (—OH), an alkoxide (—OR), water (—OH₂⁺), and an alcohol (—OHR⁺), wherein R, R₂ and R₃ are each individually selected from H and C₁₋₃ alkyl and Ar is selected from unsubstituted phenyl or phenyl substituted with one or more substituents selected from halogen, C₁₋₃ alkyl, and any combination thereof; and
n is selected from 0 to 2;
(b) when n is 0 or 1, converting the compound of Formula II-a or a salt thereof to a compound of Formula I or a pharmaceutically acceptable salt thereof (e.g., by oxidizing the compound of Formula II using a suitable oxidizing agent as oxone);
(c) separating the desired isomer of a compound of Formula I or a pharmaceutically acceptable salt thereof (e.g., using chiral chromatography); and
(d) optionally purifying the desired isomer of a compound of Formula I or a pharmaceutically acceptable salt thereof (e.g., by using one or more suitable solvents as described herein).

In a second aspect, the present invention relates to a process for the chiral separation of a compound of Formula I or a pharmaceutically acceptable salt thereof, comprising:
(a) providing a solution of a racemic compound of Formula I-a or a pharmaceutically acceptable salt thereof in one or more solvents; and
(b) separating the desired isomer of a compound of Formula I or a pharmaceutically acceptable salt thereof using chiral chromatography.

In one embodiment, in step (a), the one or more solvents is selected from methanol, ethanol, 1-propanol, 2-propanol, n-butanol, isobutanol, nitromethane, chloroform, acetonitrile, acetone, MIBK, MEK, toluene, heptane, ethyl acetate, propyl acetate, n-pentyl acetate, isopropyl acetate, butyl acetate, propionitrile, diethylether, dimethylether, diisopropylether, diphenylether, MTBE, tetrahydrofuran, methyl tetrahydrofuran, 1,4-dioxane, dimethoxyethane, o-xylene, m-xylene, p-xylene, n-pentane, cyclopentane, n-hexane, cyclohexane, benzene, dichloromethane, dichloroethane, tetrachloromethane, chlorobenzene, dimethyl acetamide (DMA), dimethylformamide (DMF), water, or any combination of any of the foregoing.

In one embodiment, in step (b), the chiral chromatography is conducted in a solvent selected from dichloromethane, methanol, n-hexane, n-heptane, EtOH, IPA, THE, ACN, EtOAc, MTBE, n-butanol, or any combination of any of the foregoing, as an eluent.

In a third aspect, the present invention relates to a process for purifying a compound of Formula I or a pharmaceutically acceptable salt thereof.

In a first embodiment, the process of the third aspect comprises:
(a) providing a solution, dispersion, or slurry of a compound of Formula I or a pharmaceutically acceptable salt thereof in one or more solvents;
(b) optionally heating the reaction mass of step (a) to suitable temperature;
(c) cooling the reaction mass; and
(d) isolating the pure compound of Formula I or pharmaceutically acceptable salt thereof.

In a second embodiment, the process of the third aspect comprises:
(a) providing a dispersion or slurry of a compound of Formula I or a pharmaceutically acceptable salt thereof in a solvent (e.g., by stirring); and
(b) isolating the purified compound of Formula I or a pharmaceutically acceptable salt thereof (e.g., by filtration of the dispersion or slurry).

In a third embodiment, the process of the third aspect comprises:
(a) providing a solution, dispersion, or slurry of a compound of Formula I or pharmaceutically acceptable salt thereof in solvent;
(b) adding an anti-solvent to the reaction mass of step (a); and
(c) isolating the purified compound of Formula I or a pharmaceutically acceptable salt thereof.

In a fourth embodiment, the process of the third aspect comprises:
(a) providing a solution, dispersion, or slurry of a compound of Formula I or pharmaceutically acceptable salt thereof in one or more solvents;
(b) optionally heating the reaction mass of step (a) (e.g., to a suitable temperature);
(c) optionally cooling the reaction mass;
(d) adding one or more solvents to the reaction mass; and
(e) isolating the purified compound of Formula I or a pharmaceutically acceptable salt thereof.

In a fourth aspect, the present invention relates to a process for the preparation of a pharmaceutically acceptable salt of a compound of Formula I, the process comprising:
(a) providing a solution, dispersion, or slurry of a compound of Formula I in one or more solvents;
(b) adding an acid to the reaction mass of step (a);
(c) heating the reaction mass of step (b) (e.g., to a suitable temperature);
(d) cooling the reaction mass of step (c); and
(e) isolating the pharmaceutically acceptable salt of a compound of Formula I.

In a fifth aspect, the present invention relates to a process for preparing a substantially pure compound of Formula I, comprising:
(a) providing a solution, dispersion, or slurry of a pharmaceutically acceptable salt of a compound of Formula I in one or more solvents;
(b) adding a base to the reaction mass of step (a);
(c) heating the reaction mass of step (b) (e.g., to a suitable temperature);
(d) cooling the reaction mass; and
(e) isolating the substantially pure compound of Formula I.

In a sixth aspect, the present invention relates to a process for preparing a substantially pure compound of Formula III or a salt thereof, comprising:
(a) providing a solution, dispersion, or slurry of a compound of Formula III or a pharmaceutically acceptable salt thereof in one or more solvents;
(b) optionally heating the reaction mass of step (a) (e.g., to a suitable temperature, such as with hot ethanol);
(c) cooling the reaction mass; and
(d) isolating the substantially pure compound of Formula III or a pharmaceutically acceptable salt thereof.

In a sixth aspect, the present invention relates to a process for purifying a compound of Formula III or a salt thereof.

In a first embodiment, the process of the sixth aspect comprises:
(a) providing a dispersion or slurry of a compound of Formula III or a salt thereof in one or more solvents (e.g., by stirring); and
(b) isolating the purified compound of Formula III or a salt thereof (e.g., by filtration of the dispersion or slurry).

In a second embodiment, the process of the sixth aspect comprises:
(a) providing a solution, dispersion, or slurry of a compound of Formula III or a salt thereof in one or more solvents; and
(b) adding an anti-solvent to the reaction mass of step (a); and
(c) isolating the purified compound of Formula III or a salt thereof.

In a seventh aspect, the present invention relates to a process for preparing a substantially pure compound of Formula IV or a salt thereof, comprising:
(a) providing a solution, dispersion, or slurry of compound of Formula IV in one or more solvents;
(b) optionally heating the reaction mass of step (a) (e.g., to a suitable temperature);
(c) cooling the reaction mass; and
(d) isolating the substantially pure compound of Formula IV or a salt thereof.

In an eight aspect, the present invention relates to a process for purifying a compound of Formula IV or a salt thereof.

In a first embodiment, the eight aspect comprises:
(a) providing a solution, dispersion, or slurry of a compound of Formula IV or a salt thereof in solvent; and
(b) isolating the purified compound of Formula IV or a salt thereof (e.g., by filtration).

In one embodiment, the compound of Formula IV dissolves in the solution and impurities remain undissolved and are removed by filtration. In one embodiment, the compound of Formula IV remains undissolved in the solution and impurities dissolve and the compound of Formula IV is isolated by filtration.

In one embodiment of any of the processes described herein (such as the processes of the third, fourth, fifth, sixth, seventh or eighth aspects described herein), the solvent (or the one or more solvents) is selected from methanol, ethanol, acetonitrile, dimethyl sulfoxide, cyclohexane, dichloromethane and any combination of any of the foregoing.

In one embodiment of any of the processes described herein (such as the processes of the third, fourth, fifth, sixth, seventh or eighth aspects described herein), the solvent (or the one or more solvents) is selected from methanol, acetonitrile, dichloromethane, cyclohexane, and any combination of any of the foregoing.

In a second embodiment, the process of the eight aspect comprises:
(a) providing a solution or a dispersion of a compound of Formula IV or a salt thereof in a solvent; and
(b) adding an anti-solvent to the reaction mass of step (a); and
(c) isolating the purified compound of Formula IV or a salt thereof.

In additional embodiments of any of the processes described herein, the compound of Formula III or salt thereof has a chemical purity of at least about 90%, such as at least about 95%, at least about 98% or at least about 99%, as measured by HPLC.

In additional embodiments of any of the processes described herein, the compound of Formula IV or salt thereof has a chemical purity of at least about 90%, such as at least about 95%, at least about 98% or at least about 99%, as measured by HPLC.

In another embodiment, the compound of Formula I or a pharmaceutically acceptable salt thereof prepared by any of the processes described herein is substantially pure (i.e., is substantially free from one or more of compounds of Formulas A, B, C, D, and E).

In another embodiment, the compound of Formula I or a pharmaceutically acceptable salt thereof prepared by any of the processes described herein is substantially free from one or more of compounds of Formulas A, B, C, D, and E, wherein each such compound of Formulas A, B, C, D, and/or E is present in less than about 0.2% w/w.

In another embodiment, the compound of Formula I or a pharmaceutically acceptable salt thereof prepared by any of the processes described herein is substantially free from one or more compounds of the Formula A, B, C, D, and E.

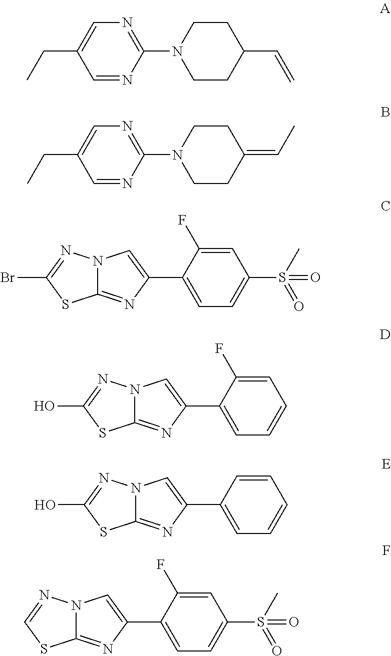

In another embodiment, the compound of Formula I or a pharmaceutically acceptable salt thereof (or any of its intermediates) prepared by any of the processes described herein is isolated from the reaction mixture using techniques such as, but not limited to, extraction, evaporation, distillation, centrifugation, filtration or by scraping, or by shaking the container, removal of the solvent including using a rotational distillation device such as a Buchi rotavapor, spray drying, agitated thin film drying, freeze drying (lyophilization), and the like, or any other techniques specific to the equipment used.

In one embodiment of any of the processes described herein, a cooling step involves cooling from any higher temperature to about 0° C., depending upon the requirement of the reaction step.

In one embodiment of any of the processes described herein, a cooling step involves cooling from room temperature to about 0° C., depending upon the requirement of the reaction step.

In one embodiment of any of the processes described herein, a cooling step involves cooling from room temperature to about 10° C.

In certain embodiments of any of the processes described herein, the pharmaceutically acceptable salt of a compound of Formula I (or any of its intermediates) is selected from a hydrochloride, a hydrobromide, a sulphate, a phosphate, an acetate, a succinate, a tartrate, a fumarate, a formate, an oxalate, a (S)-(+)-2-methoxy-2-(1-naphthyl) propionic acid salt, a chiral phthalic acid salt, a chiral dichlorophthalic acid salt, a (−)-malic acid salt, a (−)-mandelic acid salt, and a (+)-camphor-10-sulfonic acid salt.

In additional embodiments of any of the processes described herein, the pharmaceutically acceptable salt of a compound of Formula I (or any of its intermediates) is selected from a hydrochloride, a hydrobromide, a tartrate, a fumarate, a formate, an oxalate, a (S)-(+)-2-methoxy-2-(1-naphthyl) propionic acid salt, a chiral phthalic acid salt, a chiral dichlorophthalic acid salt, a (−)-malic acid salt, a (−)-mandelic acid salt, and a (+)-camphor-10-sulfonic acid salt.

In one embodiment, the compound of Formula I or a pharmaceutically acceptable salt thereof prepared by any process described herein is an amorphous form or any crystalline form or any combination of any of the foregoing in any weight percent content. In one embodiment, the compound of Formula I or a pharmaceutically acceptable salt thereof prepared by any process described herein is amorphous. In one embodiment, the compound of Formula I or a pharmaceutically acceptable salt thereof prepared by any process described herein is crystalline.

In one embodiment of any of the processes described herein, the preparation of a compound of Formula I or a pharmaceutically acceptable salt thereof is performed in situ without isolation of intermediates.

In additional embodiments of any of the processes described herein, the compound of Formula I or a pharmaceutically acceptable salt thereof has a chemical purity of at least about 90%, such as at least about 95%, at least about 98%, at least about 99%, or at least bout 99.9%, as measured by HPLC.

In additional embodiments of any of the processes described herein, the compound of Formula I or a pharmaceutically acceptable salt thereof has a enantiomeric purity of at least about 90%, such as at least about 95%, at least about 98%, at least about 99%, or at least bout 99.9%, as measured by HPLC.

In additional embodiments of any of the processes described herein, the compound of Formula I or a pharmaceutically acceptable salt thereof has an enantiomeric excess of at least about 90%, such as at least about 95%, at least about 98%, at least about 99%, or at least bout 99.9%, as measured by HPLC.

In additional embodiments of any of the processes described herein, the compound of Formula I or a pharmaceutically acceptable salt thereof has an isomeric purity of at least about 97%, such as at least about 99%, at least about 99.5%, or at least about 99.9%.

In additional embodiments of any of the processes described herein, the compound of Formula I or a pharmaceutically acceptable salt thereof is characterized by a particle size distribution wherein d₉₀ is about 0.1 μm to about 200 μm.

In additional embodiments of any of the processes described herein, the compound of Formula I or a pharmaceutically acceptable salt thereof is characterized by a particle size distribution wherein d₉₀ is about 2 μm to about 150 μm.

In additional embodiments of any of the processes described herein, the compound of Formula I or a pharmaceutically acceptable salt thereof is characterized by a particle size distribution wherein d₉₀ is less than about 100 μm.

In additional embodiments of any of the processes described herein, the compound of Formula I or a pharmaceutically acceptable salt thereof is characterized by a particle size distribution wherein d₉₀ is less than about 60 μm, d₅₀ is less than about 20 μm and d₁₀ is less than about 10 μm.

Scheme 1 shows an exemplary process for the preparation of a compound of Formula I or a pharmaceutically acceptable salt thereof.

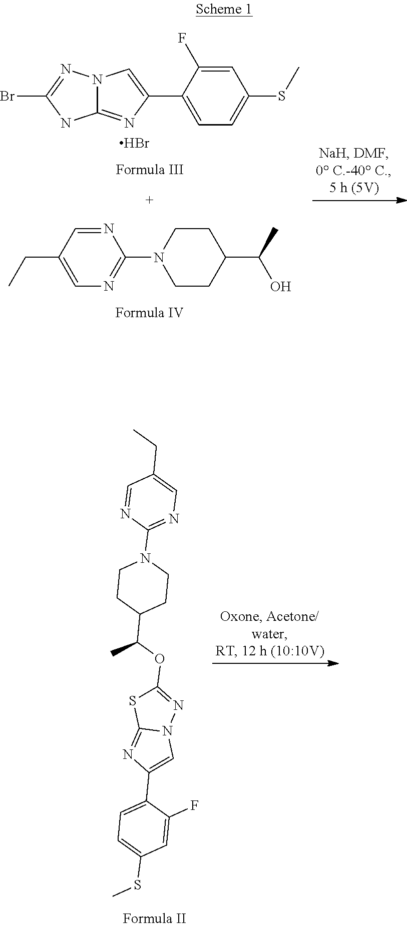

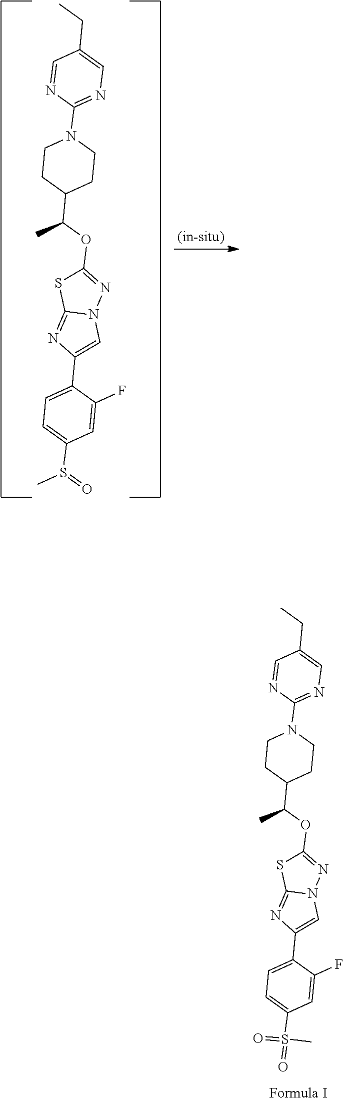

Scheme 2 shows an exemplary process for the preparation of a compound of Formula III or a pharmaceutically acceptable salt thereof.

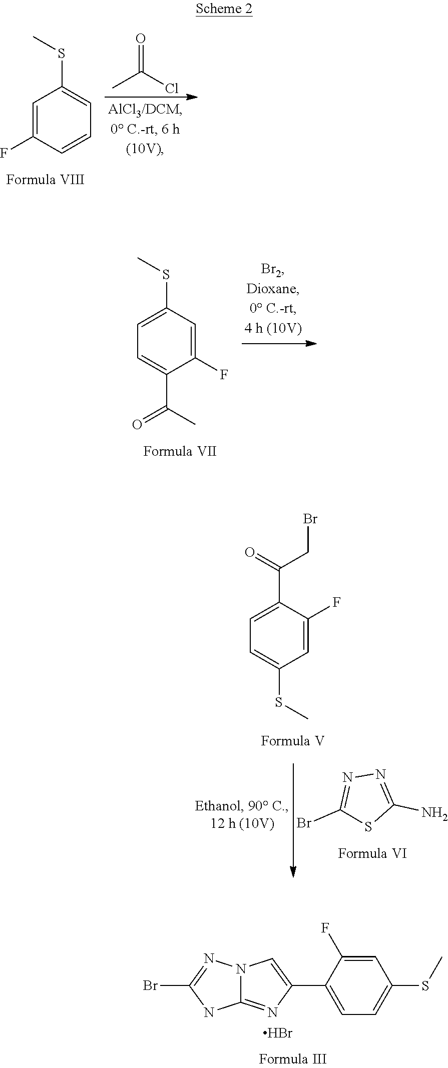

Scheme 3 shows an exemplary process for the preparation of a compound of Formula IV or a pharmaceutically acceptable salt thereof.

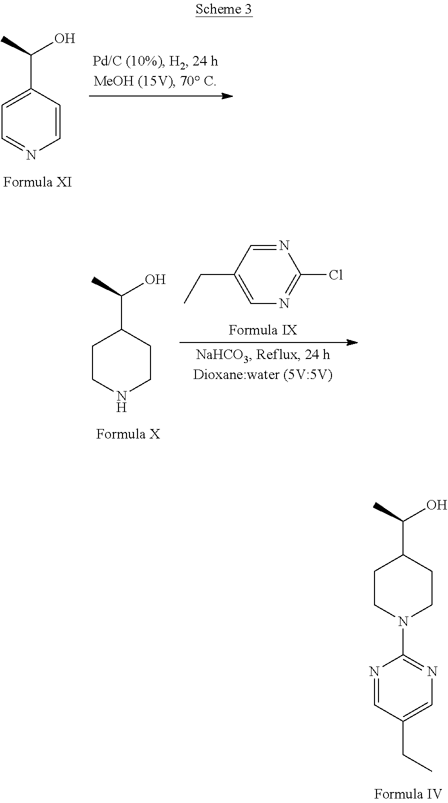

Scheme 4 shows another exemplary process for the preparation of a compound of Formula I or a pharmaceutically acceptable salt thereof.

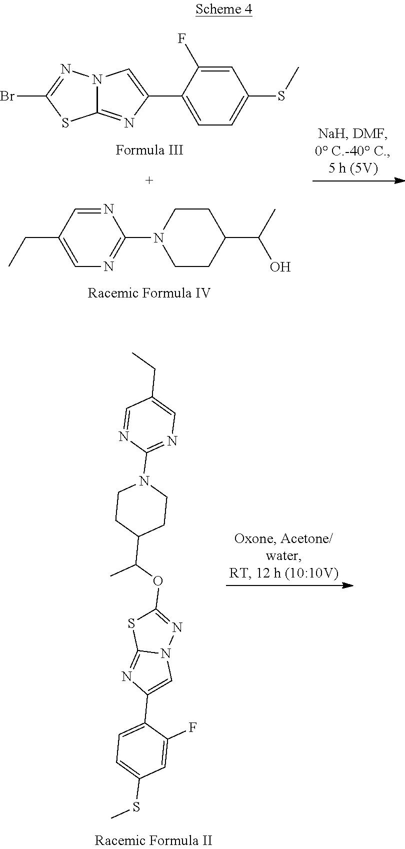

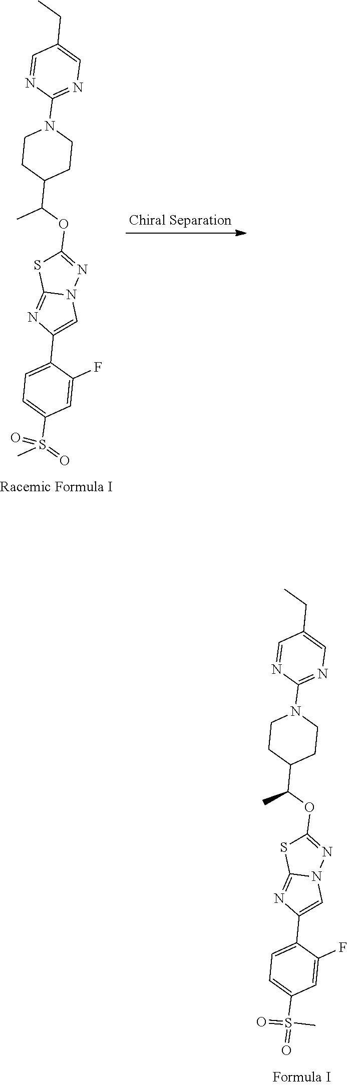 [SMILES: CCc1cnc(N2CCC(C(C)Oc3nn4cc(-c5ccc(S(C)(=O)=O)cc5F)nc4s3)CC2)nc1 ; CCc1cnc(N2CCC([C@H](C)Oc3nn4cc(-c5ccc(S(C)(=O)=O)cc5F)nc4s3)CC2)nc1]

Certain specific aspects and embodiments of the present invention will be explained in greater detail with reference to the following examples, which are provided only for purposes of illustration and should not be construed as limiting the scope of the application in any manner. Variations of the described procedures, as will be apparent to those skilled in the art, are intended to be within the scope of the present application.

### Examples

Particle size analysis was performed using a Malvern particle size analyzer (Mastersizer 3000, Malvern Instrument Ltd).

### Sample Preparation

Weigh accurately 100 mg of sample in 100 mL beaker. Add 3 drops of Tween-80 and 1 mL of water and make a paste with the help of glass rod. Then add 10 ml water and sonicate externally for 10 seconds with continued shaking.

### Instrument Parameters

| Dispersant | Water | |
|---|---|---|
| Dispersant Refractive Index | 1.330 | |
| Level Sensor Threshold | 100 | |
| Particle Type | Non spherical | |
| Scattering Model | Mie | |
| Material Name | Compound of Formula I | |
| | (e.g., compound of Formula IA) | |
| Particle Refractive Index | 0.250 | |
| Particle Absorption Index | 0.001 | |
| Particle Density | 0.85 | g/cm³ |
| Background measurement duration | 6 | seconds |
| Sample measurement duration | 6 | seconds |
| Delay between measurements | 0 | seconds |
| Pre-measurement delay | 0 | seconds |

| Obscuration Limit | 10-20% |
|---|---|
| Stirrer RPM | 2000 |
| Analysis Model | General purpose |
| Analysis sensitivity | Normal |
| Fine Mode | Off |

### Procedure

After cleaning, initialize the system and measure background. Add sample to reach obscuration between 10%-20% and wait for stable obscuration. Start the analysis or run the standard operating procedure as per given instrumental parameters. Annalize the sample in duplicate and report the average result (instrument average) of two preparations at D₁₀, D₅₀, and D₉₀.

### Example 1: Preparation of a Compound of Formula X

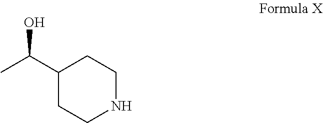

A solution of (1S)-1-(4-pyridinyl) ethanol (200 g, 1.626 mol, 99% ee) in methanol (3 L) was charged with Pd/C (40 g) under a nitrogen atmosphere. The mixture was evacuated and purged with hydrogen several times and then stirred under an atmosphere of hydrogen for 24 h at 60° C. On completion, the reaction mixture was filtered through celite bed and washed with methanol (3.0 L). All organic solvent was removed under reduced pressure to yield (IR)-1-(4-piperidinyl) ethanol (200.0 g, 95.0%) as off white solid.

Chemical Purity: 95% by Titration

### Titration Procedure:

Accurately weigh and transfer about 200 mg of test sample into a clean and dry 100.0 mL dried titration vessel. Add 50.0 mL of glacial acetic acid.

Titrate against 0.1 M perchloric acid by potentiometrically and record the reading (V).

Similarly perform the blank titration by omitting the test sample and record the reading (B).

### Instrument Parameters

| | |
|---|---|
| Sensor | DGi113-SC |
| Sensor Type | pH |
| Unit | mV |
| Stir Speed | 40% |
| Stir Duration | 70 seconds |
| Mode | Acid/base (Non aq.) |
| Threshold | 10 mV/mL |

Specific Optical Rotation (1% solution in dichloromethane): −16.835°

### Example 2: Preparation of a Compound of Formula VII

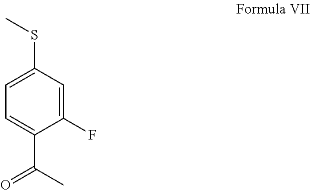

A round bottom flask was charged dichloromethane (100 mL). AlCl₃ (11.26 g, 84.50 mmol) was added at 0° C. in one portion and the resulting solution was allowed to stir for 45 min. Acetyl chloride (5.77 mL, 80.98 mmol) was added dropwise and stirred for 1 h at the same temperature. To this was added a solution of Formula VIII (10.0 g, 70.42 mmol) in dichloromethane (10 mL) slowly at 0° C. The mixture was then stirred for 3 h at RT. Progress of reaction was monitored by TLC. On completion, the reaction mass was poured into ice cold water (200 mL), then extracted with dichloromethane (200 mL). The organic layer was dried over Na₂SO₄ and evaporated under reduced pressure. The crude product was washed with cyclohexane (20 mL) to obtain the title compound as a light brown solid (10.0 g, 77%), Melting Point: 61-62° C.

Chemical Purity: 99.86% by HPLC

### Chromatographic Conditions

| Column | Symmetry C-18; (750 × 4.6) mm; 3.5 μm | |
|---|---|---|
| Column Oven Temperature | 35° C. | |
| Auto Sampler Temperature | 25° C. | |
| Flow Rate | 0.7 mL per minute | |
| Injection Volume | 10 μL | |
| Wavelength | 305 nm | |
| Run Time | 25 minutes | |
| Mode | Gradient | |
| | RT (Min) | RRT |
| Retention Time | About 14.0 | 1.00 |

### Example 3: Preparation of a Compound of Formula V

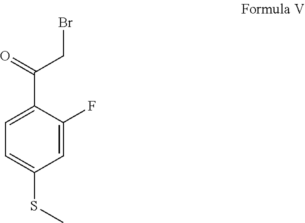

To a stirred solution of Formula VII (20.0 g, 108.6 mmol) in dioxane (200 mL) was added a solution of bromine (5.61 mL, 108.6 mmol) in dioxane (200 mL) slowly at room temperature (RT). The mixture was then stirred for 3 h at RT. Progress of reaction was monitored by TLC. On completion, the reaction mass was poured into ice cold water (200 mL). The solid precipitated was filtered and dried under vacuum to give the title compound (13.3 g, 78%) as a brown solid, (Melting Point. 55-59° C.).

Chemical Purity: 93.56% by HPLC

### Chromatographic Conditions

| Column | Symmetry C-18; (750 × 4.6) mm; 3.5 μm | |
|---|---|---|
| Column Oven Temperature | 35° C. | |
| Auto Sampler Temperature | 25° C. | |
| Flow Rate | 0.7 mL per minute | |
| Injection Volume | 10 μL | |
| Wavelength | 314 nm | |
| Run Time | 25 minutes | |
| Mode | Gradient | |
| | RT (Min) | RRT |
| Retention Time | About 15.0 | 1.00 |

### Example 4: Preparation of a Compound of Formula VI

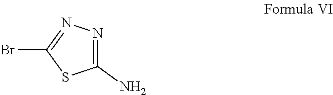

To a stirred solution of 1,3,4-thiadiazol-2-amine (5 g, 49.44 mmol) in methanol (250 mL) was added bromine (23.70 g, 148.32 mmol) dropwise at room temperature and the resulting mixture was stirred for 3 h. Completion of reaction was monitored by TLC. The reaction mixture was evaporated under reduced pressure. After addition of water to the reaction mass, a solid precipitated out which was isolated by filtration. The solid was washed with water and dried under vacuum to obtain the title compound of Formula VI (6.5 g, 72.95%) as a yellow solid.

### Example 5: Preparation of a Compound of Formula IV

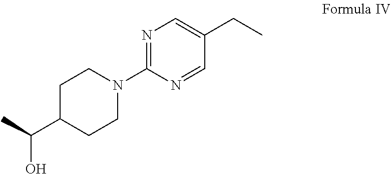 [SMILES: CCc1cnc(N2CCC([C@H](C)O)CC2)nc1]

To a solution of compound of Formula X (200.0 g, 1.550 mol) in 1, 4-dioxane (1.0 L) and water (1.0 L) was added a compound of Formula IX (shown below) (210.0 g, 1.472 mol) at room temperature. NaHCO₃ (390.6 g, 4.651 mol) was then added to the reaction mixture and refluxed at 100° C. for 16 h. Upon completion, the reaction mass was cooled to 20° C., water (2.0 L) was added, and the product extracted with EtOAc (2.0 L×3). The combined organic layer dried over Na₂SO₄ then concentrated under reduced pressure to obtain the compound of Formula IV (500.0 g crude) as a light-yellow sticky solid.

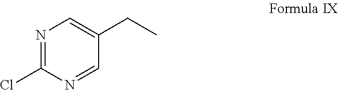

### Example 6: Purification of a Compound of Formula IV

The light-yellow sticky solid (500 g) obtained in Example 5 was purified by stirring with cyclohexane (1.0 L) at 10° C. The solid was filtered and dried to obtain the compound of Formula IV (320.0 g, 88.0%) as off white solid.

Chemical Purity: 99.6% by HPLC

### Chromatographic Conditions

Use a Ghost Buster column (e.g., Welch Materials, Inc.) with dimension 50×4.6 mm and HPLC tubing of length 6.5 cm. Install the ghost buster after the in-line filter and before the injection in water HPLC system.

| Column | X-Bridge C-18; (150 × 4.6) mm; 5 μm | |
|---|---|---|
| Column Oven Temperature | 25° C. | |
| Auto Sampler Temperature | 10° C. | |
| Flow Tate | 1.0 mL per minute | |
| Injection Volume | 10 μL | |
| Wavelength | 250 nm | |
| Needle Wash | Use acetonitrile and water in the ratio | |
| | of 90:10% with extended mode | |
| Run Time | 60 minutes | |
| Mode | Gradient | |
| Rinsing Solvent | Tetrahydrofuran (Use Extended mode) | |
| | RT (Min) | RRT |
| Retention Time | About 11.0 to 14.0 | 1.00 |

Isomeric Purity (enantiomeric excess): 99.32% (ee 98.64%)

Specific Optical Rotation (1% solution in dichloromethane): +5.9°

### Example 7: Preparation of a Compound of Formula III

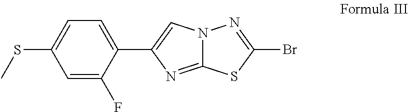 [SMILES: CSc1ccc(-c2cn3nc(Br)sc3n2)c(F)c1]

To a stirred solution of a compound of Formula V (20.0 g, 76.33 mmol) in ethanol (200 mL) was added a compound of Formula VI (13.66 g, 76.33 mmol) and the resulting reaction mass was refluxed at 90° C. for 24 h. Upon completion, the reaction mass was cooled to 50° C., filtered and dried to obtain a crude compound of Formula III.

### Example 8: Purification of a Compound of Formula III

The crude compound of Formula III obtained in Example 7 was washed with hot ethanol then dried under vacuum to obtain a compound of Formula III (20.0 g, 77%) as a brown solid.

Chemical Purity: 98.69% by HPLC

### Chromatographic Conditions

Use a Ghost Buster column (e.g., Welch Materials, Inc.) with dimension 50×4.6 mm and HPLC tubing of length 6.5 cm. Install the ghost buster after the in-line filter and before the injection in water HPLC system.

| Column | X-Bridge C-18; (150 × 4.6) mm; 5 μm | |
|---|---|---|
| Column oven temperature | 25° C. | |
| Auto sampler temperature | 5° C. | |
| Flow rate | 1.0 mL per minute | |
| Injection volume | 10 μL | |
| Wavelength | 280 nm | |
| Needle wash | Use THF and methanol in the ratio of | |
| | 80:20% with extended mode | |
| Run time | 60 minutes | |
| Mode | Gradient | |
| Rinsing Solvent | Tetrahydrofuran (Use Extended mode) | |
| | RT (Min) | RRT |
| Retention Time | About 35.1 | 1.00 |

### Example 9: Preparation of a Compound of Formula II

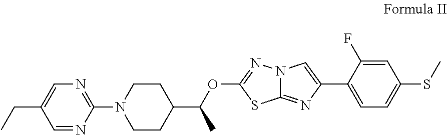 [SMILES: CCc1cnc(N2CCC([C@H](C)Oc3nn4cc(-c5ccc(SC)cc5F)nc4s3)CC2)nc1]

To a stirred solution of a compound of Formula IV (1 kg, 4.249 mol) in dimethylformamide was added a compound of Formula III (1.75 kg, 4.116 mol) at 25-30° C., then the reaction mass was cooled to 10° C. Sodium hydride (0.43 kg, 10.75 mol) was then added lot wise within 45 minutes while maintaining an internal temperature below 35° C. The reaction mass was stirred for 3 hours at 35-40° C. Upon completion, the reaction mass was poured into ice cold water (15 L). The resulting solid precipitate was isolated by filtration, washed with water (10 L) and dried under vacuum overnight. The solid material obtained was stirred with MeOH (7.5 L) for 1 h and filtered. This process was repeated (2×5 L MeOH) to obtain the compound of Formula II (1.3 kg, 70%) as an off-white solid.

Chemical Purity: 99.6% by HPLC

| Column | X-Bridge C-18; (150 × 4.6) mm; 5 μm | |
|---|---|---|
| Column Oven Temperature | 25° C. | |
| Auto Sampler Temperature | 10° C. | |
| Flow Rate | 1.0 mL per minute | |
| Injection Volume | 10 μL | |
| Wavelength | 250 nm | |
| Needle Wash | Use mixture of HPLC grade THF and | |
| | acetonitrile in the ratio of 95:5% v/v | |
| Run Time | 50 minutes | |
| Mode | Gradient | |
| | RT (Min) | RRT |
| Retention Time | About 29.4 | 1.00 |

Isomeric Purity (enantiomeric excess): 99.82% (ee 99.64%)

Specific Optical Rotation (1% solution in dichloromethane): −26.2°

### Example 10: Preparation of a Compound of Formula I

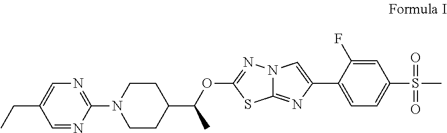 [SMILES: CCc1cnc(N2CCC([C@H](C)Oc3nn4cc(-c5ccc(S(C)(=O)=O)cc5F)nc4s3)CC2)nc1]

To a stirred solution of a compound of Formula II (0.75 kg, 1.5151 mol) in acetone (7.5 L) was added a solution of oxone (potassium peroxymonosulfate) (1.25 kg, 4.090 mol) in water (7.5 L) slowly at 0° C., maintaining a temperature below 25° C. The resulting mixture was stirred at 25° C. for 3 h. Upon completion, cold water (15 L) was added to the reaction mass followed by addition of ethylacetate (7.5 L) and the resulting mixture was stirred for 30 min. The organic layer was separated and the aqueous layer again extracted with ethylacetate (2×7.5 L) and separated. The combined organic layer was dried over Na₂SO₄ then concentrated under reduced pressure to give the compound of Formula I (1.0 kg, crude) as a light-yellow solid.

### Example 11: Purification of a Compound of Formula I

Methanol (5.25 L) was added to the crude product of Formula I (1.0 Kg) obtained in example 10 and the resulting reaction mass stirred for 1 h. The solid precipitate was filtered and washed with methanol (0.6 L). The wet cake obtained was again stirred with methanol (2.25 L), then filtered and dried to give a compound of Formula I (0.7 Kg) as an off-white solid. The dried solid was then taken in acetonitrile (4.2 L). The resulting slurry was stirred for 3 hours at 27-33° C., then filtered and washed with acetonitrile (1 L). The resultant solid material was dried and again stirred with methanol (3.5 L) at 25-30° C. for 1 h. The solid formed was filtered, washed with methanol (0.56 L, 0.8 V) and dried in a vacuum tray dryer (VTD) at 55-60° C. to give a compound of Formula I (0.52 Kg, 67%) as an off-white solid.

Chemical Purity: 99.86% (HPLC)

### Chromatographic Conditions

Use a Ghost Buster column (e.g., Welch Materials, Inc.) with dimension 50×4.6 mm and HPLC tubing of length 6.5 cm. Install the ghost buster after the in-line filter and before the injection in water HPLC system.

| Column | X-Bridge C-18; (150 × 4.6) mm; 5 μm | |
|---|---|---|
| Column Oven Temperature | 45° C. | |
| Auto Sampler Temperature | 10° C. | |
| Flow Rate | 1.0 mL per minute | |
| Injection Volume | 10 μL | |
| Wavelength | 245 nm | |
| Run Time | 50 minutes | |
| Mode | Gradient | |
| Rinsing Solvent | Tetrahydrofuran (Use Extended mode) | |
| | RT (Min) | RRT |
| Retention Time | About 19.4 | 1.00 |

Isomeric Purity (enantiomeric excess): 99.98% (ee 99.96%)

Specific Optical Rotation (1% solution in dichloromethane): −25.8°

### Example 12: Preparation of a Compound of Racemic Formula II-b

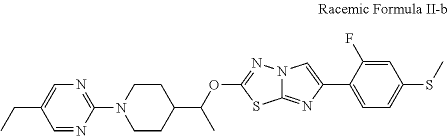 [SMILES: CCc1cnc(N2CCC(C(C)Oc3nn4cc(-c5ccc(SC)cc5F)nc4s3)CC2)nc1]

To a stirred solution of a compound of racemic Formula IV (0.500 kg, 2.124 mol) in dimethylformamide was added a compound of Formula III (0.875 kg, 2.0575 mol) at 25-30° C., then the mixture was cooled to 10° C. Sodium hydride (0.215 kg, 5.375 mol) was then added lot wise within 45 minutes while maintaining an internal temperature below 35° C. The reaction mass was stirred for 3 hours at 35-40° C. Upon completion, the reaction mass was poured into ice cold water (7.5 L), and the resulting solid precipitate was filtered, washed with water (5 L) and dried under vacuum overnight. The solid material obtained was stirred with methanol (3.75 L) for 1 h and filtered. This process was repeated (2×5 L MeOH) to afford a compound of racemic Formula II-b (0.65 Kg, 70%) as an off-white solid.

Chemical Purity: 99.37% by HPLC

| Column | X-Bridge C-18; (150 × 4.6) mm; 5 μm | |
|---|---|---|
| Column Oven Temperature | 25° C. | |
| Auto Sampler Temperature | 10° C. | |
| Flow Rate | 1.0 mL per minute | |
| Injection Volume | 10 μL | |
| Wavelength | 250 nm | |
| Needle Wash | Use mixture of HPLC grade THF and | |
| | acetonitrile in the ratio of 95:5% v/v | |
| Run Time | 50 minutes | |
| Mode | Gradient | |
| | RT (Min) | RRT |
| Retention Time | About 29.4 | 1.00 |

### Example 13: Preparation of a Compound of Racemic Formula I

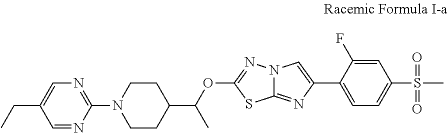 [SMILES: CCc1cnc(N2CCC(C(C)Oc3nn4cc(-c5ccc(S(C)(=O)=O)cc5F)nc4s3)CC2)nc1]

To a stirred solution of a compound of Racemic Formula II-b (0.375 kg, 0.7575 mol) in acetone (3.75 L) was added a solution of oxone (0.625 kg, 2.045 mol) in water (3.75 L) slowly at 0° C., maintaining a temperature below 25° C. The resulting mixture was stirred at 25° C. for 3 h. Upon completion, cold water (7.5 L) was added to the reaction mass followed by the addition of ethyl acetate (3.75 L). The resulting mixture was stirred for 30 min. The organic layer was separated, and the aqueous layer was again extracted with ethyl acetate (2×3.75 L) and separated. The combined organic layer was dried over Na₂SO₄, then concentrated under reduced pressure to give the compound of racemic Formula I-a (0.5 kg, crude) as a light-yellow solid. Methanol (2.5 L) was added to the crude product of racemic Formula I-a (0.5 Kg) and stirred for 1 h. The resulting solid precipitate was filtered and washed with methanol (0.3 L). The wet cake obtained was again stirred with methanol (1.1 L) filtered and dried to give the compound of Racemic Formula I-a (0.35 Kg) as an off-white solid.

Chemical Purity: 98.5% by HPLC

### Chromatographic Conditions

Use a Ghost Buster column (e.g., Welch Materials, Inc.) with dimension 50×4.6 mm and HPLC tubing of length 6.5 cm. Install the ghost buster after the in-line filter and before the injection in water HPLC system.

| Column | X-Bridge C-18; (150 × 4.6) mm; 5 μm | |
|---|---|---|
| Column Oven Temperature | 45° C. | |
| Auto Sampler Temperature | 10° C. | |
| Flow Rate | 1.0 mL per minute | |
| Injection Volume | 10 μL | |
| Wavelength | 245 nm | |
| Run Time | 50 minutes | |
| Mode | Gradient | |
| Rinsing Solvent | Tetrahydrofuran (Use Extended mode) | |
| | RT (Min) | RRT |
| Retention Time | About 19.4 | 1.00 |

### Example 14: Chiral Purification of a Compound of Racemic Formula I

The enantiomers of a compound of Racemic Formula I-a (0.35 kg) were separated by chiral prep HPLC. For this separation; Column ID-CHIRALPAK IG, 250 mm×4.6, 5 μm, mobile phase: mixture of methanol and dichloromethane in the ratio of 50:50, wavelength 305 nm was used on the basis of better peak shape and separation. The flow rate used was 1.5 ml/min, maintaining a column temperature 25° C. The eluent was concentrated under reduced pressure to give the compound of Formula I (0.15 Kg, 43%).

Chemical Purity: 99.9% by HPLC

Isomeric Purity (enantiomeric excess): 99.96% (ee 99.92%)

Specific Optical Rotation (1% solution in dichloromethane): −25.8°

All references and patent publications referenced herein are hereby incorporated by reference.

We claim:

1. A process for the preparation of a compound of Formula I or a pharmaceutically acceptable salt thereof 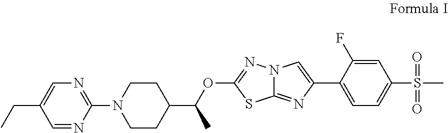 [SMILES: CCc1cnc(N2CCC([C@H](C)Oc3nn4cc(-c5ccc(S(C)(=O)=O)cc5F)nc4s3)CC2)nc1]
the process comprising:
(a) reacting a compound of Formula III-a or a salt thereof with a compound of Formula IV-a or a salt thereof to obtain a compound of Formula II-a or a salt thereof 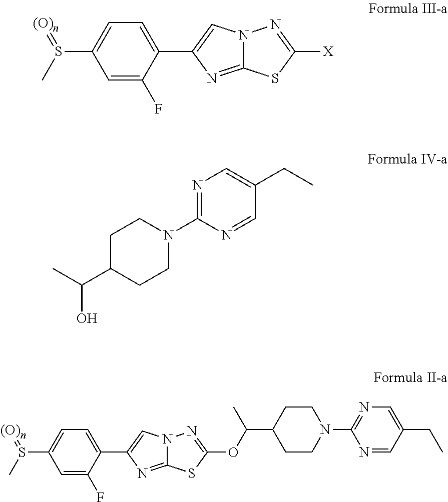
wherein X is a leaving group and n is 0, 1 or 2;
(b) when n is 0 or 1, converting the compound of formula II-a to compound of Formula I-a or pharmaceutically acceptable salt thereof 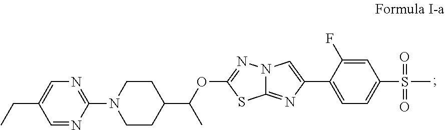 [SMILES: CCc1cnc(N2CCC(C(C)Oc3nn4cc(-c5ccc(S(C)(=O)=O)cc5F)nc4s3)CC2)nc1]
(c) separating the desired isomer of the compound of Formula I or a pharmaceutically acceptable salt thereof; and
(d) optionally purifying the desired isomer of the compound of Formula I or a pharmaceutically acceptable salt thereof.

2. A process for the preparation of a compound of Formula I or a pharmaceutically acceptable salt thereof 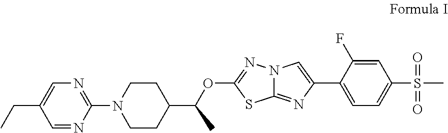 [SMILES: CCc1cnc(N2CCC([C@H](C)Oc3nn4cc(-c5ccc(S(C)(=O)=O)cc5F)nc4s3)CC2)nc1]
the process comprising:
(a) reacting a compound of Formula III-1 or a salt thereof with a compound of Formula IV or a salt thereof to obtain a compound of Formula II or a salt thereof 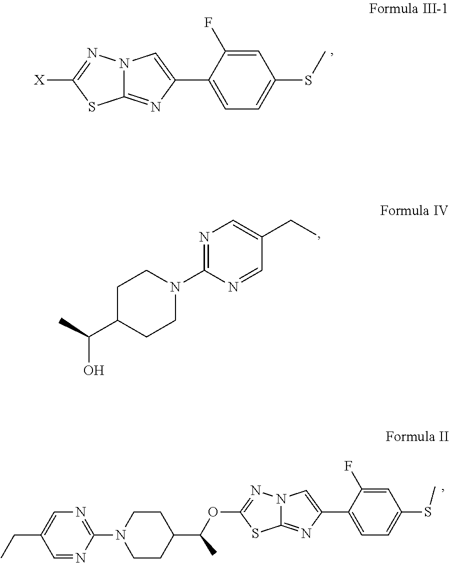
wherein X is a leaving group;
(b) converting the compound of Formula II or a salt thereof to a compound of Formula I or pharmaceutically acceptable salt thereof; and
(c) optionally purifying the compound of Formula I or a pharmaceutically acceptable salt thereof.

3. The process as claimed in claim 2, wherein the compound of Formula III-1 or a salt thereof is prepared by a process comprising:
(a) reacting a compound of Formula VIII with an acetyl halide to form a compound of Formula VII 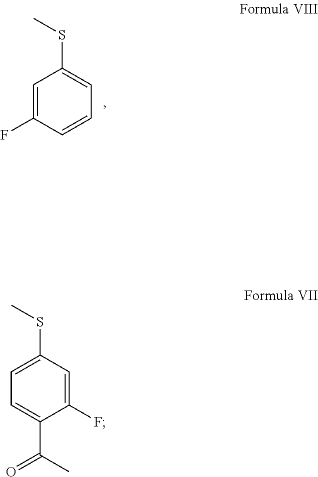
(b) converting the compound of Formula VII to a compound of Formula V-1 in the presence of solvent and a halogenating agent 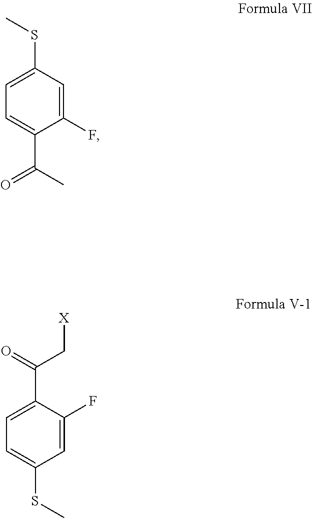
wherein X is a leaving group;
(c) reacting the compound of Formula V-1 with a compound of Formula VI-1 or a salt thereof to give a compound of Formula III-1 or salt thereof 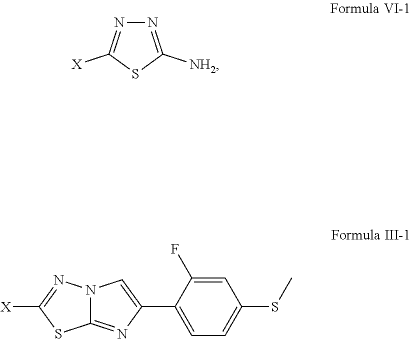
wherein X is as defined above; and
(d) optionally, purifying the compound of Formula III-1 or pharmaceutically acceptable salt thereof using one or more suitable solvents.

4. The process as claimed in claim 3, wherein the acetyl halide is selected from acetyl fluoride, acetyl chloride, acetyl bromide, acetyl iodide, and any combination thereof, preferably acetyl chloride.

5. The process as claimed in claim 3, wherein the halogenating agent is selected from fluorine, chlorine, bromine, iodine, N-chloroosuccinimide, N-bromosuccinimide, N-iodosuccinimide, hydrogen fluoride, hydrogen chloride, hydrogen bromide, hydrogen iodide, thionyl chloride, thionyl bromide, oxalyl chloride, oxalyl bromide, and any combination thereof.

6. The process as claimed in claim 2, wherein the compound of Formula IV or a salt thereof is prepared by a process comprising:
(a) reacting a compound of Formula XI with a reducing agent to give a compound of formula X 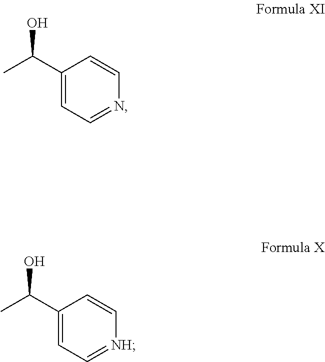
(b) reacting the compound of Formula X with a compound of Formula IX-a in the presence of solvent to obtain a compound of Formula IV 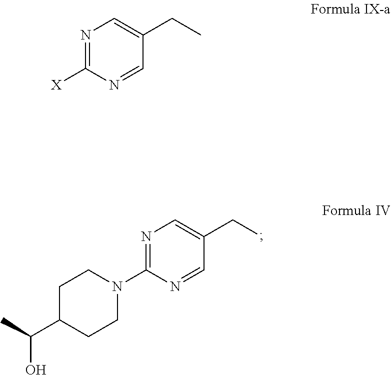
and
(c) optionally purifying the compound of Formula IV.

7. The process as claimed in claim 6, wherein the reducing agent is selected from Ni, Raney Ni, Pd/C, Pd(OH)₂, Na metal, Pt, PtO₂ and any combination thereof.

8. The process as claimed in claim 1, wherein the process comprises:
(a) reacting a hydrobromide salt of a compound of Formula III with a compound of Formula IV-a or a salt thereof to give a compound of Formula II-b or a salt thereof 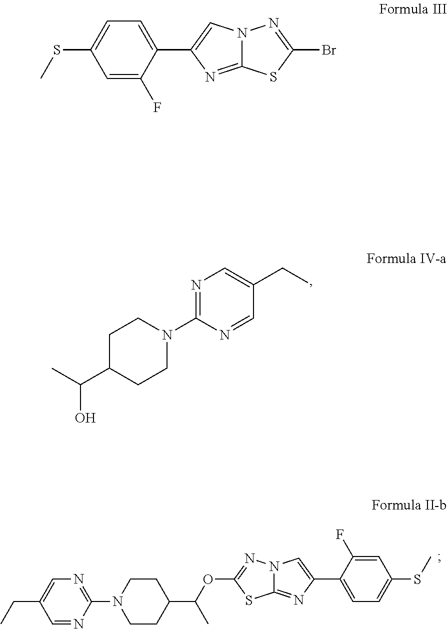
(b) converting the compound of Formula II-b or a salt thereof to a compound of Racemic Formula I-a or a pharmaceutically acceptable thereof 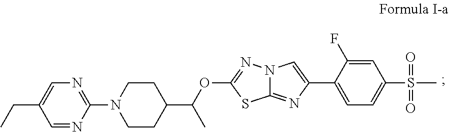 [SMILES: CCc1cnc(N2CCC(C(C)Oc3nn4cc(-c5ccc(S(C)(=O)=O)cc5F)nc4s3)CC2)nc1]
(c) separating the desired isomer of the compound of Formula I or pharmaceutically acceptable salt thereof; and
(d) optionally purifying the desired isomer of the compound of Formula I or a pharmaceutically acceptable salt thereof.

9. The process as claimed in claim 1, wherein step (c) comprises
(a) adding a chiral acid to the compound of Formula I-a or a pharmaceutically acceptable salt thereof in one or more solvents;
(b) optionally isolating the chiral acid salt of the compound of Formula I;
(c) adding a base;
(c) separating the desired isomer of compound of Formula I or a pharmaceutically acceptable salt thereof; and
(e) optionally purifying the desired isomer of the compound of Formula I or a pharmaceutically acceptable salt thereof.

10. The process according to claim 8, wherein step (c) is performed by chiral chromatography using a solvent or solvent mixture selected from dichloromethane, methanol, n-Hexane, n-heptane, EtOH, IPA, THF, ACN, EtOAc, MTBE, n-butanol, and any combination of any of the foregoing as an eluent.

11. A process for the purification of a compound of Formula I or a pharmaceutically acceptable salt thereof 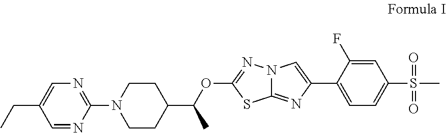 [SMILES: CCc1cnc(N2CCC([C@H](C)Oc3nn4cc(-c5ccc(S(C)(=O)=O)cc5F)nc4s3)CC2)nc1]
the process comprising:
(a) providing a solution, dispersion, or slurry of the compound of Formula I or a pharmaceutically acceptable salt thereof in one or more solvents;
(b) heating the reaction mass of step (a);
(c) cooling the reaction mass;
(d) optionally adding one or more solvents; and
(e) isolating the purified compound of Formula I or a pharmaceutically acceptable salt thereof.

12. The process according to claim 13, wherein the one or more solvents are selected from methanol, ethanol, acetonitrile, dimethyl sulfoxide, cyclohexane, dichloromethane, and any combination of any of the foregoing.

13. A compound of Formula I or a pharmaceutically acceptable salt thereof 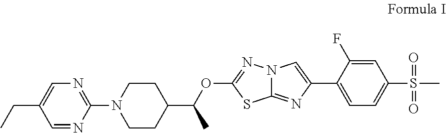 [SMILES: CCc1cnc(N2CCC([C@H](C)Oc3nn4cc(-c5ccc(S(C)(=O)=O)cc5F)nc4s3)CC2)nc1]
wherein the compound of Formula I or a pharmaceutically acceptable salt thereof has
(i) a chemical purity of more than about 99%,
(ii) an enantiomeric excess of more than about 99%, or
(iii) both (i) and (ii).

14. A compound of Formula I or a pharmaceutically acceptable salt thereof 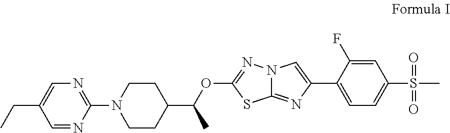 [SMILES: CCc1cnc(N2CCC([C@H](C)Oc3nn4cc(-c5ccc(S(C)(=O)=O)cc5F)nc4s3)CC2)nc1]
wherein the compound of Formula I or a pharmaceutically acceptable salt thereof is substantially free of one or more compounds of the Formulas A, B, C, D, and E

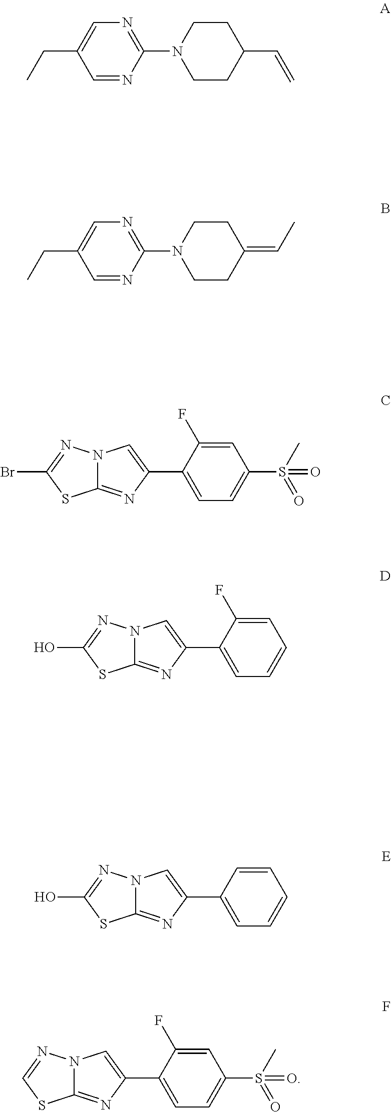

15. A compound of Formula I or a pharmaceutically acceptable salt thereof 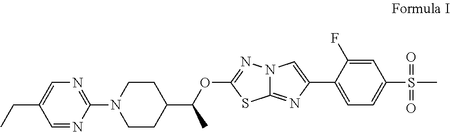 [SMILES: CCc1cnc(N2CCC([C@H](C)Oc3nn4cc(-c5ccc(S(C)(=O)=O)cc5F)nc4s3)CC2)nc1]
wherein the compound of Formula I or a pharmaceutically acceptable salt thereof is characterized by a particle size distribution wherein
(i) a d₉₀ of less than about 60 μm,
(ii) a d₅₀ of less than about 20 μm,
(iii) a d₁₀ of less than about 10 μm, or
(iv) any combination of any of (i), (ii) and (iii).
